(19) United States
(12) Plant Patent Application Publication
(10) Pub. No.: US 2008/0209605 P1
(43) Pub. Date: Aug. 28, 2008

(54) ANGELONIA PLANT NAMED 'CAS WHIT07'

(76) Inventor: Ralph T. Perkins, Gilroy, CA (US)

Correspondence Address: JONDLE & ASSOCIATES P.C., 858 HAPPY CANYON ROAD SUITE 230, CASTLE ROCK, CO, 80108, US

(21) Appl. No.: 11/709,663

(22) Filed: Feb. 22, 2007

(51) Int. Cl. A01H 5/00

(52) U.S. Cl. PLT/404

(57) ABSTRACT

An Angelonia cultivar particularly distinguished by having a white flower color, a prostrate, outwardly spreading, vigorous growth and well-branched plant habit is disclosed.

### GENUS AND SPECIES

Angelonia angustifolia

### VARIETY DENOMINATION

‘Cas Whit07’

### BACKGROUND OF THE NEW PLANT

The present invention comprises a new and distinct cultivar of Angelonia, botanically known as Angelonia angustifolia, and hereinafter referred to by the cultivar name ‘Cas Whit07’. The new cultivar originated from a hybridization made in September 2002 in Gilroy, Calif. The female parent was ‘228-1’ (unpatented) a proprietary Angelonia plant having white flowers, while the male parent was ‘197-2’, (unpatented), a proprietary Angelonia plant having white flowers.

The seeds produced by the self-pollination were sown in April 2003. A single plant selection was chosen for further evaluation and for asexual propagation in July 2003.

The new cultivar was created in September, 2002 in Gilroy, Calif. and has been asexually reproduced repeatedly by vegetative cuttings and tissue culture in Gilroy, Calif. and Andijk, The Netherlands over a three-year period. The plant has also been trialed at Gilroy, Calif., Andijk, The Netherlands and Hillscheid, Germany. The line has been shown to reproduce true to type in successive generations of asexual propagation.

Plant Breeder's Rights for this cultivar were applied for in Canada on March 21, 2006.

### SUMMARY OF THE INVENTION

The following are the most outstanding and distinguishing characteristics of the new cultivar when grown under normal horticultural practices in Gilroy, Calif. and Andijk, The Netherlands.

1. White flower color; and

2. Prostrate, outwardly spreading well-branched and vigorous plant habit.

### DESCRIPTION OF THE PHOTOGRAPHS

This new Angelonia plant is illustrated by the accompanying photographs which shows the overall plant habit, including inflorescences, buds and foliage of the plant. The plants were approximately 15 to 16 weeks old and grown in a greenhouse in Hillscheid, Germany during the summer. The colors shown are as true as can be reasonably obtained by conventional photographic procedures.

FIG. 1. shows the overall plant habit including inflorescences, buds and foliage pf the plant

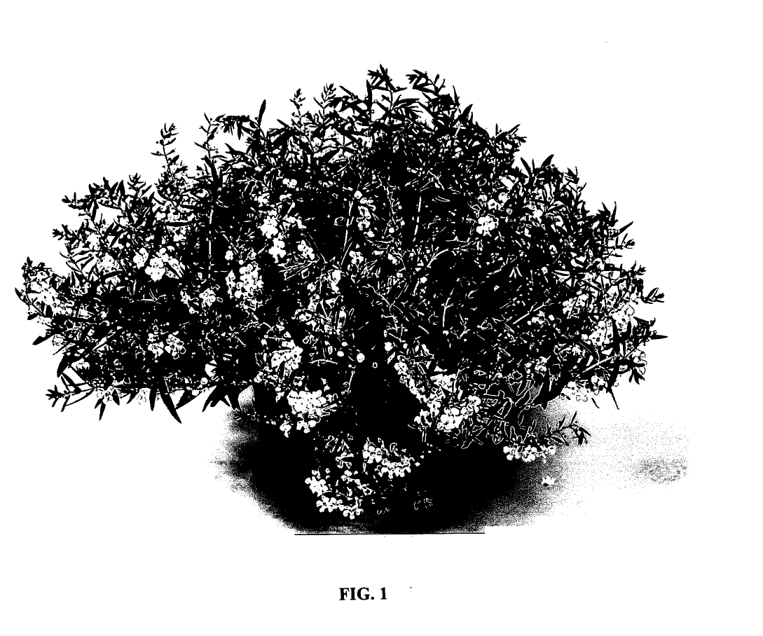

FIG. 2. shows a close-up of the mature inflorescence

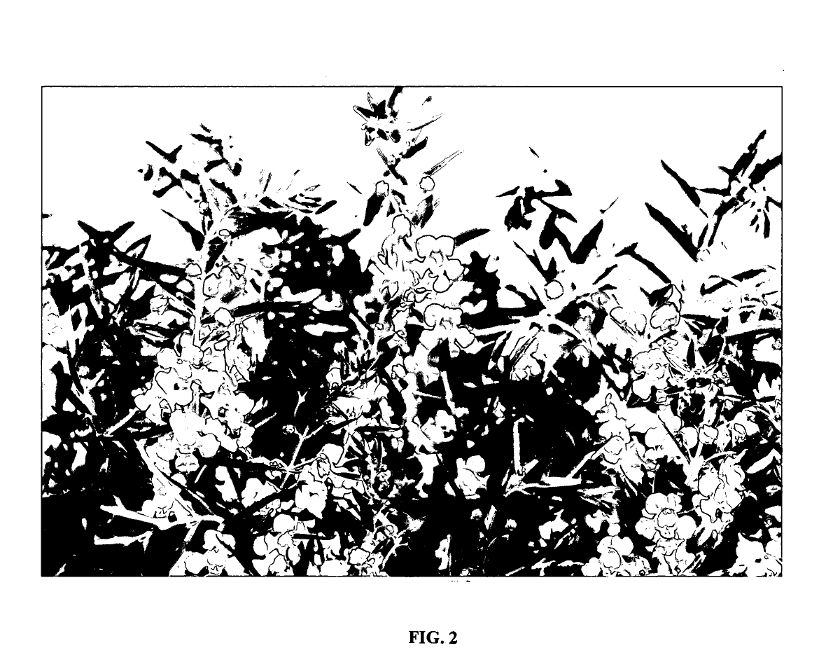

### DESCRIPTION OF THE NEW CULTIVAR

The following detailed description sets forth the distinctive characteristics of ‘Cas Whit07’. The data which define these characteristics were collected from asexual reproductions carried out in Gilroy, Calif. The detailed description was taken on 15 to 18 week old plants in 4-inch pots. The plants had one plant growth regulator treatment and one terminal pinch after cuttings were transplanted to the 4 inch pots. Color readings were taken in the late summer season under natural light in a greenhouse. Color references are primarily to the RHS Colour Chart of The Royal Horticultural Society of London (RHS), 2001 edition. Texture descriptions were viewed with a dissecting microscope.

### DETAILED BOTANICAL DESCRIPTION

Classification:
   Family.—Scrophulariaceae.
   Botanical.—Angelonia angustifolia.
   Common.—Angel flower.
Parentage:
   Male parent.—‘197-2’, a proprietary white-flowered Angelonia plant (unpatented).
   Female parent.—‘228-1’ a proprietary white-flowered Angelonia plant (unpatented).
Plant description:
   Form.—Mostly prostrate while outwardly spreading.
   Habit.—A vigorous growth habit and well-branched; pinching terminal apices 1 to 2 times after transplanting enhances branching.
   Height (measured from the base of plant).—10 cm to 14 cm.
   Width (measured from the widest portion of plant).—45 cm to 55 cm.
   Time to initiate and develop roots.—18 to 20 days.
   Root description.—Fibrous, free branching.
   Time to produce a finished flowering plant.—8 to 9 weeks from a 4-inch pot.
   Outdoor plant performance.—Plant in full sun; free flowering through the summer; some heat tolerance; use in mixed container plantings or hanging baskets.
Stems:
   Length.—35 cm to 45 cm.
   Diameter.—0.2 cm to 0.3 cm.
   Internode length.—2.5 cm to 4.0 cm.
   Color.—Between RHS 144A and RHS 144B.
   Texture.—Glandular hairs, varying in sizes.
   Anthocyanin.—Absent.
Leaves:
   Arrangement.—Opposite.
   Immature leaves.—Color: Upper surface: Closest to RHS 139A Lower surface: Closest to RHS 137A.
   Mature leaves.—Length: 6.3 cm to 6.8 cm Width: 1.1 cm to 1.3 cm Shape: Linear to lanceolate Apex: Acute Base: Fused Margin: Serrate Color: Upper surface: Closest to RHS 139A Lower surface: Closest to RHS 137A Texture: Few glandular hairs Venation: Pinnate Venation color: RHS 144B.
Flower bud:
   Shape.—Globular/round.
   Diameter.—0.3 cm to 0.5 cm.
   Length.—0.5 cm to 0.7 cm.
   Color at tight bud.—RHS 150D.
Inflorescence:
   Blooming habit.—Continuous throughout the growing season; good floriferousness.
   Lastingness of individual blooms on the plant.—6 to 9 days.
   Fragrance.—Slightly sweet.
   Inflorescence type.—Terminal raceme.
   Floret type.—Single flowers; sometimes two in a leaf axis; 5 petals fused at the base.
   Florets per raceme.—25 to 30.
   Floret diameter (at widest point).—2.3 cm to 2.5 cm.
Flower description:
   Flower color.—Upper surface: Closest to RHS 155 (White); RHS 144C at the pedicel attachment Lower surface: Closest to RHS 155 (White).
   Corolla color (inside).—Closest to RHS 155 (White) with a spot of RHS 144B to RHS 144C (fading some as the flower matures).
   Petals.—Size: Upper lip: Length: 0.9 cm to 1.0 cm (from corolla opening) Width: 1.0 cm to 1.1 cm Lower lip, lateral petal: Length: 1.8 cm to 1.9 cm from the corolla opening Width: 1.8 cm to 1.9 cm Lower lip, mid-petal: Length: 0.9 cm to 1.0 cm from the corolla opening Width: 1.0 cm Apex: Rounded Base: Fused Margin: Entire Texture: Papillose with few glandular hairs; those along the corolla opening and the lower mid-petal have yellow-green hue.
   Sepals.—Sepal number: 5, fused at the base Color: RHS 143A Length: 0.4 cm Width: 0.1 cm Shape: Lanceolate Apex: Acute Margin: Entire Anthocyanin: Absent Texture: Short glandular hairs.
   Pedicels.—Color: RHS 144B Length: 1.4 cm to 1.5 cm Diameter: 0.075 cm Texture: Glandular hairs, varying in size.
Reproductive organs:
   Stamens.—4, in pairs.
   Stamen length.—0.5 cm.
   Filament color.—Closest to RHS 155 (White).
   Filament length.—0.4 cm.
   Anther color.—Not observed.
   Pollen amount.—Moderate.
   Pollen color.—RHS N155D (White).
   Pistil.—One.
   Pistil length.—0.35 cm to 0.4 cm.
   Style length.—0.2 cm to 0.3 cm.
   Stigma color.—Closest to RHS 155 (White).
   Style color.—Closest to RHS 155 (White).
Fruit and seed set: No fruit or seed production has been observed
Disease and insect resistance: None observed

### COMPARISON WITH PARENTAL LINES AND KNOWN CULTIVARS

‘Cas Whit07’ differs from the female parent ‘228-1’ (unpatented) in that ‘Cas Whit07’ has darker green foliage, more branching, a fuller plant habit and is more floriferous than ‘228-1’.

‘Cas Whit07’ differs from the male parent ‘197-2’ (unpatented) in that ‘Cas Whit07’ has better cascading habit, larger flowers and is more floriferous than ‘197-2’.

‘Cas Whit07’ differs from the commercial cultivar ‘Balangbawi’ (U.S. Plant Pat. No. 16,555), in that ‘Cas Whit07’ has larger foliage, a more prostate plant habit, larger pedicels and is more floriferous than ‘Balangbawi’.

I claim:

1. A new and distinct Angelonia plant as shown and described herein.
